(19) United States
(12) Patent Application Publication
(10) Pub. No.: US 2024/0338867 A1
(43) Pub. Date: Oct. 10, 2024

(54) IMAGE PROCESSING APPARATUS AND NON-TRANSITORY COMPUTER-READABLE STORAGE MEDIUM

(71) Applicants: National University Corporation Tokai National Higher Education and Research System, Nagoya-shi (JP); CANON MEDICAL SYSTEMS CORPORATION, Tochigi (JP)

(72) Inventors: Toshiaki TAOKA, Nagoya-shi (JP); Yoshiki TANAKA, Chiba City (JP); Nobuyasu ICHINOSE, Otawara (JP); Takaya MORI, Nasushiobara (JP); Masanori OZAKI, Hachioji (JP)

(73) Assignees: National University Corporation Tokai National Higher Education and Research System, Nagoya-shi (JP); CANON MEDICAL SYSTEMS CORPORATION, Tochigi (JP)

(21) Appl. No.: 18/625,365

(22) Filed: Apr. 3, 2024

(30) Foreign Application Priority Data
Apr. 7, 2023 (JP) 2023-062974

(51) Int. Cl. G06T 11/20; G06T 7/00; G06T 7/11

(52) U.S. Cl. CPC G06T 11/206; G06T 7/0012; G06T 7/11; G06T 2207/10088; G06T 2207/20021; G06T 2207/20092; G06T 2207/30016

(57) ABSTRACT

An image processing apparatus according to an embodiment includes processing circuitry. The processing circuitry is configured to obtain pixel values of at least three Magnetic Resonance (MR) images of mutually-different types. The processing circuitry is configured to generate a scatter diagram in which values based on the pixel values are arranged in a region having at least three-dimensional axes and which displays a value group related to a target tissue in such a manner that a value group related to a specific target serving as a reference is always displayed in the same position, and then cause a display to display the scatter diagram.

### CROSS-REFERENCE TO RELATED APPLICATIONS

This application is based upon and claims the benefit of priority from Japanese Patent Application No. 2023-062974, filed on Apr. 7, 2023; the entire contents of which are incorporated herein by reference.

### FIELD

Embodiments described herein relate generally to an image processing apparatus and a non-transitory computer-readable storage medium.

### BACKGROUND

Conventionally, in the medical field, diagnosing processes are performed on various types of diseases, by using medical images such as Magnetic Resonance (MR) images taken by a Magnetic Resonance Imaging (MRI) apparatus.

Generally speaking, in a diagnosing process using MR images, the diagnosing process is performed by taking various types of MR images. In that situation, an image interpreting doctor interprets images for pathological characteristics and a range of progression while constructing, in his/her mind, information obtained from the plurality of types of MR images being taken and analysis data.

### BRIEF DESCRIPTION OF THE DRAWINGS

FIG. 1 is a diagram illustrating an exemplary configuration of an image processing apparatus according to a first embodiment;

FIG. 2 is a flowchart illustrating an example of a processing procedure in processes performed by processing functions included in processing circuitry of the image processing apparatus according the first embodiment;

FIG. 3 is a drawing illustrating an example of an operation screen displayed by a display controlling function according to the first embodiment;

FIG. 4 is a drawing illustrating examples of processes performed by the display controlling function according to the first embodiment;

FIG. 5 is a drawing illustrating another example of the processes performed by the display controlling function according to the first embodiment;

FIGS. 6A and 6B are a drawing illustrating examples of processes performed by a display controlling function according to a second embodiment; and

FIG. 7 is a drawing illustrating examples of processes performed by a display controlling function according to a third embodiment.

### DETAILED DESCRIPTION

An image processing apparatus according to an embodiment includes an obtaining unit and a display controlling unit. The obtaining unit is configured to obtain pixel values of at least three MR images of mutually-different types. The display controlling unit is configured to generate a scatter diagram in which values based on the pixel values are arranged in a region having at least three-dimensional axes and which displays a value group related to a target tissue in such a manner that a value group related to a specific target serving as a reference is always displayed in the same position, and then cause a display to display the scatter diagram.

Exemplary embodiments of an image processing apparatus and a non-transitory computer-readable storage medium will be explained in detail below, with reference to the accompanying drawings.

### First Embodiment

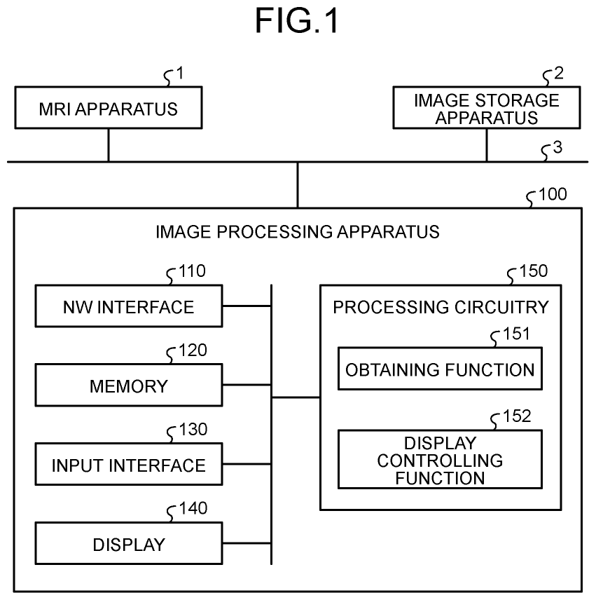

FIG. 1 is a diagram illustrating an exemplary configuration of an image processing apparatus according to a first embodiment.

For example, as illustrated in FIG. 1, an image processing apparatus 100 according to the present embodiment is connected to an MRI apparatus 1 and to an image storage apparatus 2 via a network 3, so as to be able to communicate with each other. In addition, the image processing apparatus 100 may further be connected to one or more other apparatuses (not illustrated) via the network 3.

The MRI apparatus 1 is configured to take images of an examined subject (hereinafter, “patient”) by using a magnetic resonance phenomenon. More specifically, the MRI apparatus 1 is configured to acquire magnetic resonance data from the patient, by executing various types of imaging sequences on the basis of image taking conditions set by an operator. Further, the MRI apparatus 1 is configured to generate two—or three-dimensional MR images by performing an image processing process such as a Fourier transform process on the acquired magnetic resonance data.

The image storage apparatus 2 is configured to store therein the MR images acquired by the MRI apparatus 1. For example, the image storage apparatus 2 is realized by using a computer machine such as a server of a Picture Archiving and Communication System (PACS) and is configured to store therein the MR images in a format compliant with a Digital Imaging and Communication in Medicine (DICOM) scheme.

The image processing apparatus 100 is configured to process the MR images related to the patient. More specifically, the image processing apparatus 100 is configured, via the network 3, to obtain the MR images either from the MRI apparatus 1 or the image storage apparatus 2 and to process the obtained MR images. For example, the image processing apparatus 100 is realized by using a computer machine such as a workstation.

For example, the image processing apparatus 100 includes a network (NW) interface 110, a memory 120, an input interface 130, a display 140, and processing circuitry 150.

The NW interface 110 is configured to control transfer of various types of data and communication performed between the image processing apparatus 100 and other apparatuses via the network 3. More specifically, the NW interface 110 is connected to the processing circuitry 150 and is configured to transmit any of the data received from the other apparatuses to the processing circuitry 150 and to transmit any of the data received from the processing circuitry 150 to any of the other apparatuses. For example, the NW interface 110 is realized by using a network card, a network adapter, a Network Interface Controller (NIC), or the like.

The memory 120 is configured to store therein various types of data and various types of programs. More specifically, the memory 120 is connected to the processing circuitry 150 and is configured to store therein data received from the processing circuitry 150 and to read and transmit any of the data stored therein to the processing circuitry 150. For example, the memory 120 is realized by using a semiconductor memory element such as a Random Access Memory (RAM) or a flash memory, or a hard disk, an optical disk, or the like.

The input interface 130 is configured to receive operations to input various types of instructions and various types of information from the operator. More specifically, the input interface 130 is connected to the processing circuitry 150 and is configured to convert the input operations received from the operator into electrical signals and to transmit the electrical signals to the processing circuitry 150. For example, the input interface 130 is realized by using a trackball, a switch button, a mouse, a keyboard, a touch-pad on which input operations can be performed by touching an operation screen thereof, a touch-screen in which a display screen and a touch-pad are integrally formed, a contactless input interface using an optical sensor, an audio input interface, and/or the like. Further, in the present disclosure, the input interface 130 does not necessarily need to include physical operation component parts such as the mouse, the keyboard, and/or the like. For instance, possible examples of the input interface 130 include electrical signal processing circuitry configured to receive an electrical signal corresponding to an input operation from an external input mechanism provided separately from the apparatus and to transmit the electrical signal to controlling circuitry.

The display 140 is configured to display various types of information and various types of data. More specifically, the display 140 is connected to the processing circuitry 150 and is configured to display the various types of information and the various types of data received from the processing circuitry 150. For example, the display 140 is realized by using a liquid crystal monitor, a Cathode Ray Tube (CRT) monitor, a touch-panel, or the like.

The processing circuitry 150 is configured to perform various types of processes by controlling constituent elements included in the image processing apparatus 100. For example, the processing circuitry 150 is configured to perform the various types of processes in accordance with the input operations received from the operator via the input interface 130. Further, for example, the processing circuitry 150 is configured to store data received by the NW interface 110 from other apparatuses, into the memory 120. Further, for example, the processing circuitry 150 is configured to transmit data to any of the other apparatuses, by transmitting the data read from the memory 120 to the NW interface 110. In addition, for example, the processing circuitry 150 is configured to cause the display 140 to display any of the data read from the memory 120.

A configuration of the image processing apparatus 100 according to the present embodiment has thus been explained. The image processing apparatus 100 according to the present embodiment structured as described above has a function to perform an analyzing process to assist diagnosing processes using MR images.

Generally speaking, in a diagnosing process using MR images, the diagnosing process is performed by taking various types of MR images. In that situation, an image interpreting doctor interprets images for pathological characteristics and a range of progression while constructing, in his/her mind, information obtained from the plurality of types of MR images being taken and analysis data.

In such a diagnosing process using MR images, for the purpose of making it easier for the image interpreting doctor to perform the diagnosing process, for example, one idea is to configure a display to display a scatter diagram generated by arranging values based on the plurality of types of MR images in a region having a plurality of axes.

However, when such a scatter diagram is displayed, the values used on the axes of the scatter diagram are not absolute values like CT values and do not have a reference level. As a result, the distribution of the values varies depending on individual patients and the pixel values. For this reason, there is a problem where it is difficult to make observations of common characteristics among cases of different patients or chronological changes in treatment effects of an individual patient.

To cope with the circumstances described above, the image processing apparatus 100 according to the present embodiment has a function of facilitating making observations of common characteristics among cases of different patients and chronological changes in treatment effects of an individual patient.

More specifically, in the present embodiment, the processing circuitry 150 of the image processing apparatus 100 includes an obtaining function 151 and a display controlling function 152. In the present example, the obtaining function 151 is an example of the obtaining unit. Further, the display controlling function 152 is an example of the display controlling unit.

The obtaining function 151 is configured to obtain pixel values of at least three MR images of mutually-different types. Further, the display controlling function 152 is configured to generate and to cause the display 140 to display a scatter diagram in which values based on the pixel values obtained by the obtaining function 151 are arranged in a region having at least three-dimensional axes and which displays a value group related to a target tissue in such a manner that a value group related to a specific target serving as a reference is always displayed in the same position. In this situation, the display 140 is an example of the display.

In the present embodiment, the processing circuitry 150 is realized by using a processor, for example. In that situation, processing functions included in the processing circuitry 150 are stored in the memory 120 in the form of computer-executable programs, for example. Further, the processing circuitry 150 is configured to realize the functions corresponding to the programs by reading and executing the programs stored in the memory 120. In other words, the processing circuitry 150 that has read the programs has the processing functions illustrated in FIG. 1.

With this configuration, the scatter diagram displaying the value group related to the target tissue is displayed in such a manner that the value group related to the specific target serving as a reference is always displayed in the same position. As a result, it is possible to objectively express a distribution of the values related to the target tissue in the scatter diagram. Consequently, in the present embodiment, it is possible to easily make observations of common characteristics among cases of different patients and chronological changes in treatment effects of an individual patient.

Next, processes performed by the processing functions included in the processing circuitry 150 of the image processing apparatus 100 according to the present embodiment will be explained in detail.

In the following sections, an example will be explained in which the display controlling function 152 is configured to generate, as the scatter diagram, a ternary graph having three-dimensional axes. Also, in the following sections, an example will be explained in which the specific target serving as a reference is a specific tissue. The specific tissue will be referred to as a “landmark”.

In other words, the display controlling function 152 is configured to generate and to cause the display 140 to display the ternary graph in which the values based on the pixel values obtained by the obtaining function 151 are arranged in a region having three-dimensional axes and which displays the value group related to the target tissue in such a manner that a value group related to the landmark is always displayed in the same position.

Further, in the following sections, an example will be explained in which the display controlling function 152 is configured to set three landmarks and to display value groups related to the landmarks in the positions at vertices of the ternary graph.

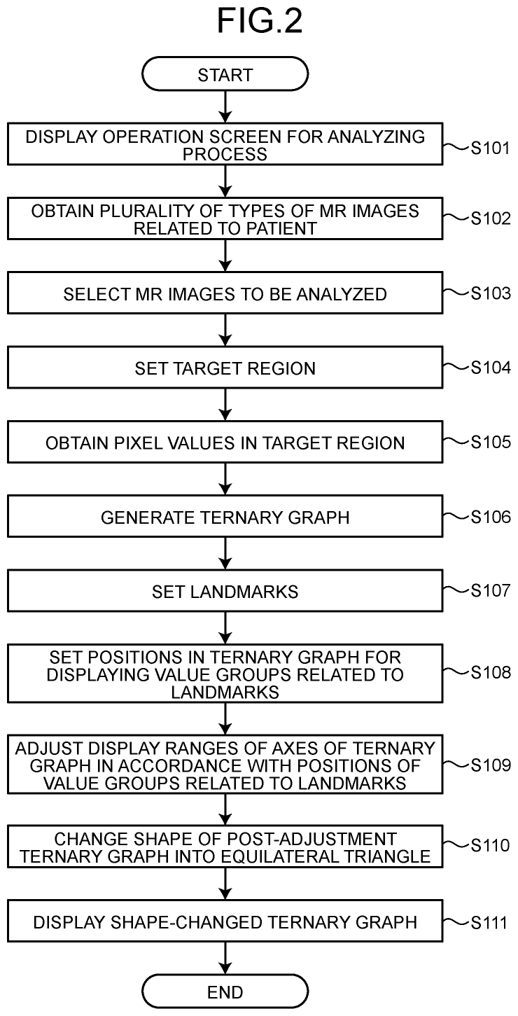

FIG. 2 is a flowchart illustrating an example of a processing procedure in processes performed by the processing functions included in processing circuitry 150 of the image processing apparatus 100 according the first embodiment.

For example, as illustrated in FIG. 2, in the present embodiment, to begin with, the display controlling function 152 causes the display 140 to display an operation screen for an analyzing process (step S101).

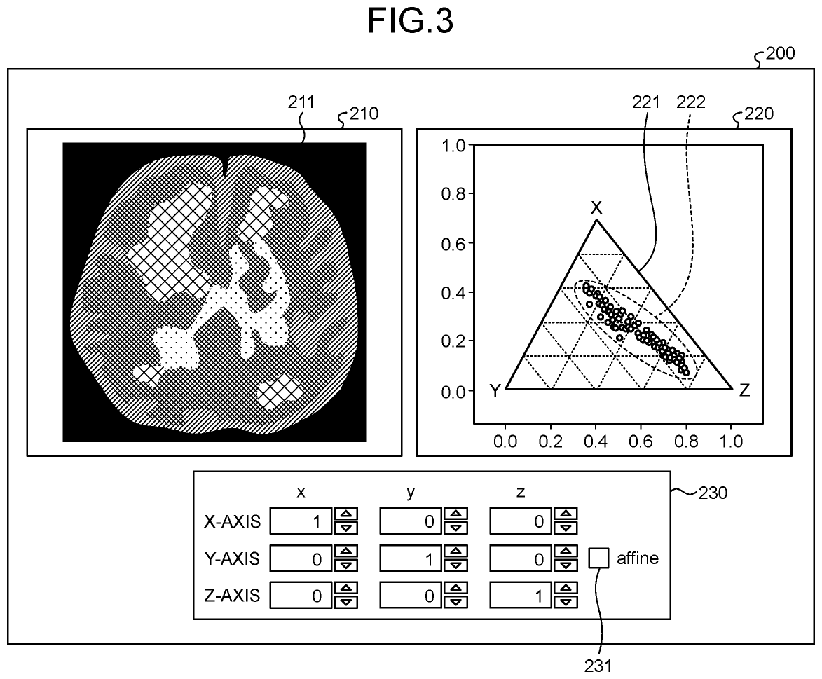

FIG. 3 is a drawing illustrating an example of the operation screen displayed by the display controlling function 152 according to the first embodiment.

For example, as illustrated in FIG. 3, in response to a request from the operator, the display controlling function 152 causes the display 140 to display an operation screen 200 including: an image display region 210 for displaying an MR image; a scatter diagram display region 220 for displaying the ternary graph; and an operation panel 230 for inputting a maximum value and a minimum value for each of the axes of the ternary graph.

Returning to the description of FIG. 2, the obtaining function 151 subsequently obtains the plurality of types of MR images related to the patient subject to a diagnosing process (step S102).

For example, the obtaining function 151 receives, from the operator, an operation to designate the patient subject to the diagnosing process and obtains, either from the MRI apparatus 1 or the image storage apparatus 2, the plurality of types of MR images related to the patient designated by the operator. In this situation, the plurality of types of MR images obtained by the obtaining function 151 may be three-dimensional images (volume data) or may be a plurality of two-dimensional images (multi-slice data) or a single two-dimensional image (since-slice data).

For example, as the MR images, the obtaining function 151 obtains any of the following: a T2-Weighted image (T2WI), a T1-Weighted image (TIWI), a Fluid Attenuated Inversion Recovery (FLAIR) image, a T2*-Weighted image (T2*WI), an Oscillating Gradient Spin Echo (OGSE)-b0 image, an OGSE-Diffusion Weighted image (DWI), an OGSE-Apparent Diffusion Coefficient (ADC) image, a Pulsed Gradient Spin Echo (PGSE)-b0 image, a PGSE-DW image, a PGSE-Apparent Diffusion Coefficient (ADC) image, an OGSE/PGSE-DW image, a PGSE/OGSE-ADC image, a T2 map image, and the like. Possible types of the MR images obtained by the obtaining function 151 are not limited to the examples listed above. It is also acceptable to obtain other types of MR images. Further, in addition to the MR images, one or more other images taken by another modality such as an X-ray Computed Tomography (CT) apparatus may further be obtained.

After that, the obtaining function 151 selects, from among the obtained plurality of types of MR images, three MR images of mutually-different types as MR images to be analyzed (step S103).

For example, the obtaining function 151 generates and causes the display 140 to display a list of the obtained plurality of types of MR images and reduced images and further receives, from the operator, an operation to designate the three MR images from among the MR images included in the displayed list or the reduced images. After that, the obtaining function 151 selects the MR images designated by the operator from among the plurality of types of MR images, as the MR images to be analyzed.

In this situation, for example, the obtaining function 151 may select the MR images to be analyzed in accordance with a pathological factor subject to the diagnosing process. In that situation, for example, the memory 120 may have stored therein, with respect to a plurality of pathological factors, information indicating three types of MR images suitable for the diagnosing process, for each of the pathological factors. Further, the obtaining function 151 receives, from the operator, an operation to designate the pathological factor subject to the diagnosing process and specifies the three types of MR images suitable for the diagnosing process on the designated pathological factor, by referring to the information stored in the memory 120. After that, the obtaining function 151 selects, from among the plurality of types of MR images, the MR images of the specified types, as the MR images to be analyzed. In this situation, the obtaining function 151 may obtain the pathological factor subject to the diagnosing process from an electronic chart system or the like via the network 3, instead of from the operator.

Subsequently, in the MR images to be analyzed, the display controlling function 152 sets a target region subject to the diagnosing process (step S104).

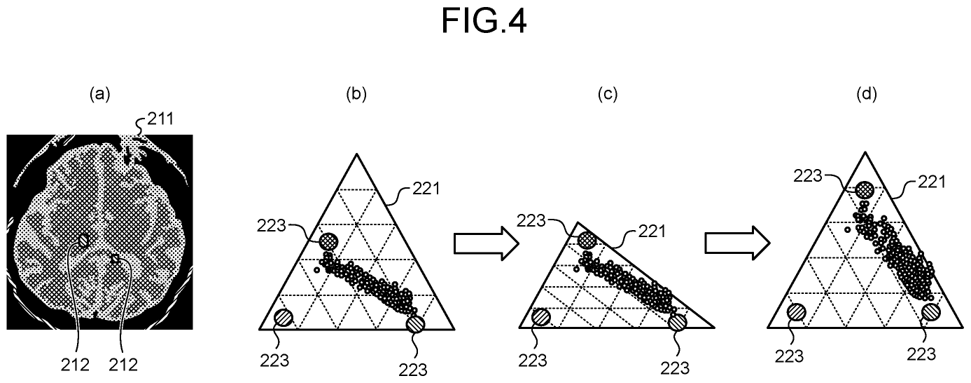

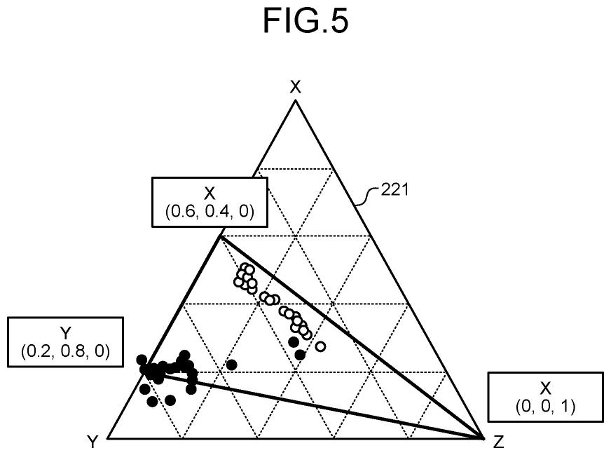

For example, as illustrated in FIG. 3, the display controlling function 152 displays, in the image display region 210 on the operation screen 200, one MR image 211 out of the three MR images selected by the obtaining function 151 as the MR images to be analyzed. After that, the display controlling function 152 receives, from the operator, an operation to designate one or more regions of interest within the MR image 211 displayed in the image display region 210. FIGS. 4 and 5 are drawings illustrating examples of processes performed by the display controlling function 152 according to the first embodiment.

For example, as illustrated in a portion (a) of FIG. 4, the display controlling function 152 receives, from the operator, an operation to designate regions of interest 212 within the MR image 211 displayed in the image display region 210. After that, on the basis of the regions of interest 212 designated by the operator, the display controlling function 152 sets a target region in the MR image 211 displayed in the image display region 210. Further, on the basis of the regions of interest 212 destinated by the operator, the display controlling function 152 also sets a target region in each of the other two MR images selected as the MR images to be analyzed. In this situation, the quantity of the regions of interest 212 designated by the operator, i.e., the quantity of the target regions set by the display controlling function 152 may be one or may be two or more.

Returning to the description of FIG. 2, the obtaining function 151 subsequently obtains the pixel values in the target region in each of the three MR images to be analyzed (step S105).

Subsequently, the display controlling function 152 generates a ternary graph having arranged therein values based on the pixel values in the target region in each of the three MR images to be analyzed that were obtained by the obtaining function 151 (step S106).

For example, as illustrated in FIG. 3, the display controlling function 152 generates the ternary graph 221 in which the values based on the pixel values of the three MR images to be analyzed are arranged in a region having three-dimensional axes X, Y, and Z and which displays a value group 222 related to the target tissue. The display controlling function 152 further displays the generated ternary graph 221 in the scatter diagram display region 220 on the operation screen 200. In this situation, the values based on the pixel values may be the pixel values themselves obtained by the obtaining function 151 or may be values obtained by processing the pixel values by performing normalization or the like.

For example, the display controlling function 152 generates and displays, in the scatter diagram display region 220 on the operation screen 200, the ternary graph 221 having arranged therein values related to an OGSE-ADC image, a PGSE/OGSE-ADC image, and a DW image (b=0).

In this situation, for example, when a plurality of target regions are set, the display controlling function 152 may display the values based on the pixel values obtained from the target regions so as to be superimposed together on the single ternary graph 221. Alternatively, for example, the display controlling function 152 may display a plurality of ternary graphs, by arranging plots based on the pixel values obtained from the target regions, so as to be each displayed in a different one of the ternary graphs respectively corresponding to the target regions.

After that, the display controlling function 152 sets the landmarks, which are the specific tissues each serving as a reference (step S107).

For example, within the MR image 211 displayed in the image display region 210, the display controlling function 152 may receive, from the operator, an operation to designate three regions of interest different from the regions of interest 212 in the target region, and may further set three landmarks on the basis of the regions of interest designated by the operator.

In another example, the display controlling function 152 may set landmarks in accordance with a purpose of the observation. In that situation, for example, the memory 120 has stored therein information indicating, with respect to each of a plurality of observation purposes, a specific tissue serving as a reference suitable for an observation of the target region. Further, by receiving, from the operator, an operation to designate an observation purpose and referring to the information stored in the memory 120, the display controlling function 152 specifies the tissue kept in correspondence with the designated observation purpose and thus sets the specified tissue as a landmark.

For example, in accordance with a site subject to the diagnosing process, the display controlling function 152 sets three tissues having mutually-different tissue characteristics each as a landmark. In this situation, it is desirable to configure the display controlling function 152 so as to set the three tissues that have the tissue characteristics different from one another, as the landmarks.

For example, when the site subject to the diagnosing process is the brain, the display controlling function 152 sets cerebrospinal fluid in the lateral cerebral ventricle, an eyeball, and the thalamus each as a landmark. In this situation, for example, the display controlling function 152 sequentially displays a slice rendering the cerebrospinal fluid in the lateral cerebral ventricle, a slice rendering the eyeball, and a slice rendering the thalamus, by switching, in accordance with an instruction from the operator, the display in the image display region 210 among the plurality of slices included in the MR images 211 of the head of the patient. Accordingly, the display controlling function 152 receives, from the operator, operations to designate, from within the slices, the cerebrospinal fluid in the lateral cerebral ventricle, the eyeball, and the thalamus each as a region of interest.

After that, as illustrated in a portion (b) of FIG. 4, for example, the display controlling function 152 displays value groups 223 respectively corresponding to the three landmarks having been set, in the ternary graph 221 displayed in the scatter diagram display region 220 on the operation screen 200.

After that, the display controlling function 152 sets positions in the ternary graph for displaying the value groups related to the landmarks (step S108).

More specifically, the display controlling function 152 sets the positions in the ternary graph 221 for displaying the value groups 223 related to the landmarks, by setting the coordinates of the vertices of the ternary graph 221 in such a manner that the positions of the vertices of the ternary graph 221 match the positions of the value groups 223 respectively related to the three landmarks that were set. In this situation, the position matched with each of the vertices of the ternary graph 221 may be the position of a single point representing each of the value groups 223 related to the corresponding landmark or may be the position of the center of gravity of the distribution of the group.

For example, as illustrated in FIG. 3, via the operation panel 230 on the operation screen 200, the display controlling function 152 receives, from the operator, an operation to input the coordinates (x, y, z) of the vertices with respect to the X-axis, the Y-axis, and the Z-axis, in such a manner that the positions of the vertices of the ternary graph match the positions of the value groups 223 respectively related to the three landmarks. After that, the display controlling function 152 sets the coordinates of the vertices of the ternary graph 221, on the basis of the coordinates (x, y, z) of the vertices input by the operator.

In this situation, let us discuss an example in which the operator inputs the coordinates of the vertices as indicated below, while the values on the axes of the ternary graph 221 are normalized to be in the range of 0 to 1, for example:

$$the X - axis : ( x , y , z ) = ( 0.6 , 0.4 , 0 )$$

$$the Y - axis : ( x , y , z ) = ( 0.2 , 0.8 , 0 )$$

$$the Z - axis : ( x , y , z ) = ( 0 , 0 , 1 )$$

In that situation, for example, as illustrated in FIG. 5, the region of the ternary graph 221 defined by the input coordinates of the vertices forms a triangle in a skewed shape having the sides of unequal lengths.

Returning to the description of FIG. 2, the display controlling function 152 subsequently generates a ternary graph displaying a value group related to the target tissue in such a manner that the value groups related to the landmarks are always displayed in the same positions, by adjusting the axes of the ternary graph so that the value groups related to the land marks are always displayed in the same positions.

More specifically, the display controlling function 152 adjusts display ranges of the axes of the ternary graph generated on the basis of the pixel values of the MR images so as to match the positions of the value groups related to the landmarks (step S109).

Even more specifically, the display controlling function 152 adjusts the display range of each of the axes of the ternary graph 221, so that the coordinates of each of the vertices set in the positions of the value groups 223 respectively related to the three landmarks are equal to the maximum value and the minimum value of the corresponding axis.

In this situation, for example, the display controlling function 152 controls the adjustments on the display ranges of the axes of the ternary graph, so that the ternary graph maintains a triangular form.

For example, in response to a change made to the display range of one of the axes of the ternary graph 221, the display controlling function 152 changes the display ranges of the other axes so that the ternary graph 221 maintains a triangular form. In other words, when the display range of one of the axes of the ternary graph 221 has been changed, the display ranges of the other axes are automatically changed so that the ternary graph 221 maintains a triangular form.

Further, for example, as illustrated in a portion (c) of FIG. 4, in the scatter diagram display region 220 on the operation screen 200, the display controlling function 152 displays the ternary graph 221 of which the display ranges of the axes were adjusted so that the positions of the vertices thereof match the positions of the value groups 223 respectively related to the three landmarks.

After that, the display controlling function 152 generates a ternary graph displaying the value group related to the target tissue so that the value groups related to the landmarks are always displayed in the same position, by changing the shape of the post-adjustment ternary graph into an equilateral triangle (step S110).

For example, as illustrated in FIG. 4(d), the display controlling function 152 changes the shape of the ternary graph 221 of which the display ranges of the axes were adjusted, into an equilateral triangle. For example, by performing an affine transformation, the display controlling function 152 changes the shape of the ternary graph 221 into the equilateral triangle. As a result, the shape of the ternary graph 221 which was skewed due to the changing of the display ranges of the axes is changed to the equilateral triangle, so that the value groups 223 related to the three landmarks are always displayed in the positions of the vertices of the ternary graph 221.

After that, in the scatter diagram display region 220 on the operation screen 200, the display controlling function 152 displays the generated shape-changed ternary graph (step S111).

For example, as illustrated in FIG. 3, when receiving, from the operator, an operation to input a check in a checkbox 231 included in the operation panel 230 on the operation screen 200, the display controlling function 152 displays, in the scatter diagram display region 220, the ternary graph 221 of which the shape was changed into the equilateral triangle. On the contrary, when receiving, from the operator, an operation to uncheck the checkbox 231 included in the operation panel 230, the display controlling function 152 displays, in the scatter diagram display region 220, the ternary graph 221 having the shape before being changed to the equilateral triangle.

In this situation, the display controlling function 152 may save the generated shape-changed ternary graph into the memory 120 so that, in response to a request from the operator, the ternary graph saved in the memory 120 is read and displayed on the display 140.

In another example, the display controlling function 152 may save, in the memory 120, adjustment values for the coordinates of the vertices and the like used at the time of generating the shape-changed ternary graph, may further read the adjustment values saved in the memory 120 in response to a request from the operator, and re-generate the shape-changed ternary graph from the ternary graph before the shape change by using the read adjustment values, so as to cause the display 140 to display the re-generated ternary graph. In this situation, when the target region or any of the landmarks is changed by the operator, the display controlling function 152 is configured to update the display of the ternary graph in a real-time manner by performing the processes described above again on the basis of the post-change target region or landmark.

An example of the processes performed by the processing functions of the processing circuitry 150 has thus been explained. In this situation, as explained earlier, when the processing circuitry 150 is realized by using one or more processors, the processes at the steps illustrated in FIG. 2, for example, are realized as a result of the processing circuitry 150 reading and executing the corresponding programs from the memory 120.

For example, the processes at steps S102, S103, and S105 are realized as a result of the processing circuitry 150 reading and executing the program corresponding to the obtaining function 151 from the memory 120. Further, for example, the processes at steps S101, S104, and S106 to S111 are realized as a result of the processing circuitry 150 reading and executing the program corresponding to the display controlling function 152 from the memory 120.

As explained above, in the first embodiment, the obtaining function 151 is configured to obtain the pixel values of the three MR images of the mutually-different types. Further, the display controlling function 152 is configured to generate and to cause the display 140 to display the ternary graph in which the values based on the pixel values obtained by the obtaining function 151 are arranged in the region having the three-dimensional axes and which displays the value group related to the target tissue in such a manner that the value groups related to the landmarks are always displayed in the same positions.

With this configuration, the ternary graph displaying the value group related to the target tissue is displayed in such a manner that the value groups related to the landmarks are always displayed in the same positions. Consequently, it is possible to objectively express, in the ternary graph, the distribution of the values related to the target tissue. As a result, in the first embodiment, it is possible to easily make observations of common characteristics among cases of different patients and chronological changes in treatment effects of an individual patient. In addition, it is possible to predict histological characteristics regarding an unknown lesion, which is helpful in narrowing down differential diagnoses.

The first embodiment has thus been explained. In addition, it is also acceptable to carry out the embodiment described above, by modifying the configuration of the image processing apparatus 100 as appropriate. Thus, in the following sections, modification examples of the first embodiment will be explained as other embodiments. In the following embodiments, differences from the first embodiment will primarily be explained, and detailed explanations of duplicate features will be omitted.

### Second Embodiment

For example, as a second embodiment, the display controlling function 152 may be configured to divide the ternary graph into a plurality of zones, to further assign mutually-different pieces of identification information to the zones, and to cause the display 140 to display an MR image on which the pieces of identification information assigned to the zones are superimposed in such a manner that, with respect to each of the zones, the position in the MR image corresponding to the value group displayed in the ternary graph is identifiable.

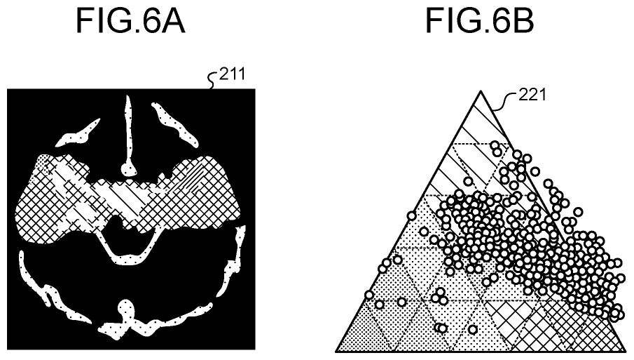

FIGS. 6A and 6B are a drawing illustrating examples of processes performed by the display controlling function 152 according to the second embodiment.

For example, as illustrated in FIGS. 6A and 6B, the display controlling function 152 is configured, similarly to the first embodiment, to display the MR image 211 in the image display region 210 included in the operation screen 200 for an analysis purpose and to display the ternary graph 221 in the scatter diagram display region 220.

In this situation, in the present embodiment, for example, as illustrated in FIG. 6B, the display controlling function 152 is configured to divide the ternary graph 221 into a plurality of triangular zones and to assign a different one of various colors to each of the different zones. Further, for example, as illustrated in FIG. 6A, the display controlling function 152 is configured to cause the display 140 to display the MR image 211 on which the colors assigned to the zones are superimposed in such a manner that, with respect to each of the zones, the position corresponding to the value group displayed in the ternary graph 221 is identifiable.

Further, the display controlling function 152 may be configured to generate a plurality of ternary graphs 221 so as to cause the display 140 to display the ternary graphs in one of the following manners: being arranged side by side; being switched from one to another; and being superimposed on one another. For example, the display controlling function 152 may generate ternary graphs 221 corresponding to pre-treatment and post-treatment, so as to cause the display 140 to display the ternary graphs either while being arranged side by side or while being switched between the two.

For example, the display controlling function 152 may be configured to display the plurality of ternary graphs 221 arranged side by side in the scatter diagram display region 220 on the operation screen 200. Alternatively, the display controlling function 152 may be configured to display as many tabs as the quantity of the ternary graphs 221 in the scatter diagram display region 220 on the operation screen 200 and to bring the mutually-different ternary graphs 221 into correspondence with the tabs, so as to display, in response to an operation of the operator selecting one of the tabs, the ternary graph 221 corresponding to the selected tab. In yet another example, the display controlling function 152 may be configured to display the plurality of ternary graphs 221 that are transparent and are superimposed on one another, in the scatter diagram display region 220 on the operation screen 200.

Further, the display controlling function 152 may be configured to display the MR image 211 in such a manner that the position corresponding to the value group related to the target tissue displayed in the ternary graph 221 is identifiable. For example, the display controlling function 152 may be configured to display a color, a pattern, or the like being mapped over the MR image 211, so as to indicate the position corresponding to the value group related to the target tissue displayed in the ternary graph 221.

Furthermore, the display controlling function 152 may also be configured to change the quantity of the zones in the entire or a partial range of the ternary graph 221. For example, the display controlling function 152 may be configured to divide the ternary graph 221 into a plurality of zones, in accordance with a tendency of the distribution of the values based on the pixel values obtained by the obtaining function 151. For example, in the ternary graph 221, the display controlling function 152 may have a larger number of zones in a range exhibiting a denser distribution of the values based on the pixel values and may have a smaller number of zones in a range having a sparser distribution of the values based on the pixel values.

In this situation, it is desirable to set the quantity of the zones in the ternary graph in accordance with the purpose of use. For example, when the ternary graph has a smaller number of zones, although it becomes more difficult to observe small feature value differences and structures in the MR image, operability is improved because the processing time will be shorter. On the contrary, when the ternary graph has a larger number of zones, although operability is lowered because the processing time will be longer, it becomes easier to observe small feature value differences and structures in the MR image.

As explained above, in the second embodiment, the display controlling function 152 is configured to divide the ternary graph into the plurality of zones, to further assign the mutually-different pieces of identification information to the zones, and to cause the display 140 to display the MR image on which the pieces of identification information assigned to the zones are superimposed in such a manner that, with respect to each of the zones, the position in the MR image corresponding to the value group displayed in the ternary graph is identifiable.

With this configuration, for example, when the distribution of the values in the ternary graph has changed the position thereof due to an effect of treatment being provided, it is possible to understand, at a glance, which part of the lesion in the image had the position change.

### Third Embodiment

Further, for example, as a third embodiment, the display controlling function 152 may be configured to display a ternary graph in such a manner that, with respect to each of a plurality of target tissues, a value group related to the target tissue is identifiable.

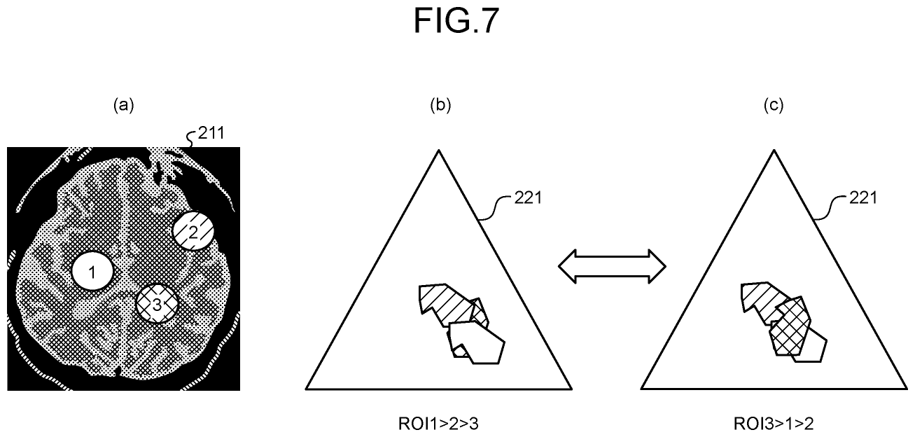

FIG. 7 is a drawing illustrating examples of processes performed by the display controlling function 152 according to the third embodiment.

For example, as illustrated in portions (a) and (b) of FIG. 7, the display controlling function 152 is configured, similarly to the first embodiment, to display the MR image 211 in the image display region 210 included in the operation screen 200 for an analysis purpose and to display the ternary graph 221 in the scatter diagram display region 220.

In this situation, in the present embodiment, for example, as illustrated in the portion (a) of FIG. 7, the display controlling function 152 is configured to receive, from the operator, an operation to designate a plurality of target tissues in the MR image 211. Further, as illustrated in portions (b) and (c) of FIG. 7, the display controlling function 152 is configured to display the ternary graph 221 in such a manner that, with respect to each of the plurality of target tissues, the value group related to the target tissue is identifiable.

For example, in the MR image 211, the display controlling function 152 is configured to display the plurality of target tissues in mutually-different colors, with mutually-different patterns, or the like. Further, the display controlling function 152 is configured to display, in the ternary graph 221, the value groups related to the target tissues, by using the same colors, the same patterns, or the like, as those of the corresponding target regions in the MR image 211.

Further, the display controlling function 152 is configured to receive, from the operator, an operation to set a priority ranking for displaying the plurality of target tissues. After that, when the positions of the value groups respectively related to the plurality of target tissues overlap with one another in the ternary graph 221, the display controlling function 152 is configured to arrange a value group related to a target tissue in a higher priority rank to be displayed closer to the viewer than the value groups related to the other target tissues.

For example, as illustrated in the portion (a) of FIG. 7, let us discuss an example in which, while three target regions (1) to (3) are set, the operator sets a priority ranking for displaying the target regions so that the target region (1) is in the highest rank, the target region (2) is in the second highest rank, and the target region (3) is in the lowest rank. In this situation, as illustrated in the portion (b) of FIG. 7, for example, the display controlling function 152 is configured to arrange, in the ternary graph 221, the value group related to the target region (1) to be displayed closest to the viewer, the value group related to the target region (2) to be displayed behind thereof, and the value group related to the target region (3) to be displayed farther behind thereof.

After that, for example, let us assume that the operator changes the priority ranking for displaying the target regions, so that the target region (3) is in the highest rank, the target region (1) is in the second highest rank, and the target region (2) is in the lowest rank. In this situation, as illustrated in the portion (a) of FIG. 7, for example, the display controlling function 152 is configured to change the displays of the groups in the ternary graph 221 so that the value group related to the target region (3) is displayed closest to the viewer, the value group related to the target region (1) is displayed behind thereof, and the value group related to the target region (2) is displayed farther behind thereof.

As explained above, in the third embodiment, the display controlling function 152 is configured to display the ternary graph in such a manner that, with respect to each of the plurality of target tissues, the value group related to the target tissue is identifiable.

With this configuration, even when the value groups respectively related to the plurality of target tissues overlap with one another in the ternary graph, it is possible to understand the positions and the shapes of the distributions of the values related to the target regions. Further, even when the value group related to any of the target regions overlaps with one or more of the value groups related to the landmarks, it is possible to visually check the positional relationship thereof.

### Modification Examples

In the embodiments described above, the example was explained in which the display controlling function 152 is configured to set the three landmarks and to display the value groups related to the landmarks in the positions of the vertices of the ternary graph; however, possible embodiments are not limited to this example. For instance, the display controlling function 152 may be configured to display the value groups related to the landmarks in positions other than the vertices of the ternary graph.

Further, in the above embodiments, the example was explained in which the display controlling function 152 is configured to generate the ternary graph having the three-dimensional axes; however, possible embodiments are not limited to this example. For instance, the display controlling function 152 may be configured to generate a scatter diagram having axes that are four or more dimensional.

Further, in the above embodiments, the examples using the MR images were explained; however, possible embodiments are not limited to these examples. For instance, it is also acceptable to use medical images taken by a modality other than the MRI apparatus such as an X-ray Computed Tomography (CT) apparatus, an ultrasound diagnosis apparatus, an X-ray diagnosis apparatus, or a Positron Emission Tomography (PET) apparatus.

### Other Embodiments

Further, the configuration of the image processing apparatus 100 explained in the above embodiments may be applied to a system intermediated by a network such as a cloud. In that situation, for example, the same processing functions as the obtaining function 151 and the display controlling function 152 described above are installed in processing circuitry provided in a server apparatus included in the system. Further, results of the processes performed by the processing functions installed in the processing circuitry of the server apparatus are transmitted to a client apparatus used by a user of the system and displayed on a display or the like provided for the client apparatus. Further, the configuration of the image processing apparatus 100 explained in the above embodiments may also be applied to a console apparatus of the MRI apparatus 1 or to the image storage apparatus 2. In that situation, for example, the same processing functions as the obtaining function 151 and the display controlling function 152 described above are installed in processing circuitry included in the console apparatus of the MRI apparatus 1 or in the image storage apparatus 2. Further, results of the processes performed by the processing functions are displayed on a display or the like provided for the console apparatus of the MRI apparatus 1 or for the image storage apparatus 2.

In the above embodiments, the processing circuitry does not necessarily need to be realized by using a single processor. It is also acceptable to structure the processing circuitry by combining together a plurality of independent processors, so that the processing functions are realized as a result of the processors executing the programs. Further, the processing functions of the processing circuitry may be realized as being distributed among or integrated into one or more pieces of processing circuitry as appropriate. Further, the processing functions of the processing circuitry may be realized by a combination of hardware such as circuitry and software. Furthermore, although the example was explained above in which the programs corresponding to the processing functions are stored in the single memory, possible embodiments are not limited to this example. For instance, it is also acceptable to store the programs corresponding to the processing functions in a plurality of memory elements in a distributed manner, so that the processing circuitry reads and executes the programs from the memory elements.

Further, in the above embodiments, the example was explained in which the obtaining unit and the display controlling unit of the present disclosure are realized by the obtaining function 151 and the display controlling function 152 of the processing circuitry 150, respectively; however, possible embodiments are not limited to this example. For instance, besides being realized by the obtaining function 151 and the display controlling function 152 described in the embodiments, the functions of the obtaining unit and the display controlling unit of the present disclosure may be realized by using hardware alone, software alone, or a combination of hardware and software.

Further, the term “processor” used in the explanation of the above embodiments denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). In this regard, instead of having the programs saved in the memory, it is also acceptable to directly incorporate the programs into the circuitry of one or more processors. In that situation, the one or more processors are configured to realize the functions by reading and executing the programs incorporated in the circuitry thereof. Further, the processors in the present embodiments do not each necessarily have to be structured as a single piece of circuitry. It is also acceptable to structure one processor by combining together a plurality of pieces of independent circuitry so as to realize the functions thereof.

In relation to the above, the programs executed by the one or more processors are provided as being incorporated, in advance, in a Read-Only Memory (ROM), one of other types of memories, or the like. The programs may be provided as being recorded on a non-transitory computer-readable storage medium such as a Compact Disk Read-Only Memory (CD-ROM), a Flexible Disk (FD), a Comact Disk Recordable (CD-R), or a Digital Versatile Disk (DVD), in a file in a format that is installable or executable by those apparatuses. Further, the programs may be stored in a computer connected to a network such as the Internet so as to be provided or distributed as being downloaded via the network. For example, the programs are structured as modules including the processing functions described above. In the actual hardware, as a result of a CPU reading and executing the programs from a storage medium such as a ROM, the modules are loaded into a main storage apparatus so as to be generated in the main storage apparatus.

Further, the constituent elements of the apparatuses illustrated in the drawings in the above embodiments are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a CPU and a program analyzed and executed by the CPU or may be realized as hardware using wired logic.

Furthermore, with regard to the processes explained in the above embodiments, it is acceptable to manually perform all or a part of the processes described as being performed automatically. Conversely, by using a publicly-known method, it is also acceptable to automatically perform all or a part of the processes described as being performed manually. Further, unless noted otherwise, it is acceptable to arbitrarily modify any of the processing procedures, the controlling procedures, specific names, and various information including various types of data and parameters that are presented in the above text and the drawings.

The various types of data handled in the present disclosure are, typically, digital data.

According to at least one aspect of the embodiments described above, it is possible to easily make observations of common characteristics among cases of different patients and chronological changes in treatment effects of an individual patient.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing apparatus, comprising:
processing circuitry configured to
obtain pixel values of at least three Magnetic Resonance (MR) images of mutually-different types; and
generate a scatter diagram in which values based on the pixel values are arranged in a region having at least three-dimensional axes and which displays a value group related to a target tissue in such a manner that a value group related to a specific target serving as a reference is always displayed in a same position, and then cause a display to display the scatter diagram.

2. The image processing apparatus according to claim 1, wherein
the processing circuitry is further configured to set the specific target and a position in the scatter diagram for displaying the value group related to the specific target, and generate the scatter diagram on a basis of the specific target and the position being set.

3. The image processing apparatus according to claim 1, wherein
the processing circuitry is further configured to set the specific target in accordance with a purpose of an observation.

4. The image processing apparatus according to claim 3, wherein
the processing circuitry is further configured to set, as the specific target, each of a plurality of targets having mutually-different tissue characteristics.

5. The image processing apparatus according to claim 4, wherein
the processing circuitry is further configured to set, as the specific target, each of cerebrospinal fluid in a lateral cerebral ventricle, an eyeball, and a thalamus.

6. The image processing apparatus according to claim 1, wherein
the processing circuitry is further configured to generate the scatter diagram displaying the value group related to the target tissue in such a manner that the value group related to the specific target is always displayed in the same position, by adjusting the axes of the scatter diagram so that the value group related to the specific target is always displayed in the same position.

7. The image processing apparatus according to claim 1, wherein
the processing circuitry is further configured to generate a ternary graph having the three-dimensional axes, as the scatter diagram.

8. The image processing apparatus according to claim 7, wherein
the processing circuitry is further configured to generate the ternary graph displaying the value group related to the target tissue in such a manner that the value group related to the specific target is always displayed in the same position, by adjusting display ranges of the axes of the ternary graph generated on a basis of the pixel values so as to fit the position of the value group related to the specific target and subsequently changing a shape of the post-adjustment ternary graph into an equilateral triangle.

9. The image processing apparatus according to claim 8, wherein
the processing circuitry is further configured to control the adjustment on the display ranges of the axes of the ternary graph so as to maintain a triangular form of the ternary graph.

10. The image processing apparatus according to claim 9, wherein
the processing circuitry is further configured to change, in response to a change made to the display range of one of the axes of the ternary graph, the display ranges of other axes, so as to maintain the triangular form of the ternary graph.

11. The image processing apparatus according to claim 1, wherein
the processing circuitry is further configured to save the scatter diagram in a storage apparatus, and, in response to a request from an operator, read the scatter diagram saved in the storage apparatus, and couse the display to display the scatter diagram.

12. The image processing apparatus according to claim 1, wherein
the processing circuitry is further configured to save an adjustment value used at the time of generating the scatter diagram in a storage apparatus, and, in response to a request from an operator, read the adjustment value saved in the storage apparatus to re-generate the scatter diagram by using the read adjustment value, and cause the display to display the re-generated scatter diagram.

13. The image processing apparatus according to claim 1, wherein
the processing circuitry is further configured to divide the scatter diagram into a plurality of zones, and assign mutually-different pieces of identification information to the zones, and cause the display to further display the MR image on which the pieces of identification information assigned to the zones are superimposed in such a manner that, with respect to each of the zones, a position in the MR image corresponding to a value group displayed in the scatter diagram is identifiable.

14. The image processing apparatus according to claim 13, wherein
the processing circuitry is further configured to generate a plurality of scatter diagrams, and cause the display to display the scatter diagrams in one of the following manners: being arranged side by side; being switched from one to another; and being superimposed on one another.

15. The image processing apparatus according to claim 13, wherein
the processing circuitry is further configured to cause the MR image to be displayed in such a manner that a position corresponding to the value group related to the target tissue displayed in the scatter diagram is identifiable.

16. The image processing apparatus according to claim 13, wherein
the processing circuitry is further configured to change a quantity of the zones in an entire or partial range of the scatter diagram.

17. The image processing apparatus according to claim 13, wherein
the processing circuitry is further configured to divide the scatter diagram into the plurality of zones in accordance with a tendency of a distribution of the values based on the pixel values.

18. The image processing apparatus according to claim 1, wherein
the processing circuitry is further configured to display the scatter diagram in such a manner that, with respect to each of a plurality of target tissues, a value group related to the target tissue is identifiable.

19. The image processing apparatus according to claim 18, wherein
the processing circuitry is further configured to receive, from the operator, an operation to set a priority ranking for displaying the plurality of target tissues, and, when positions of the value groups respectively related to the plurality of target tissues overlap with one another in the scatter diagram, arrange a value group related to a target tissue in a higher priority rank to be displayed closer to a viewer than value groups related to other target tissues.

20. The image processing apparatus according to claim 1, wherein
the specific target is a specific tissue.

21. A non-transitory computer-readable storage medium comprising a plurality of computer-executable instructions that cause a computer to execute:
obtaining pixel values of at least three Magnetic Resonance (MR) images of mutually-different types; and
generating a scatter diagram in which values based on the pixel values are arranged in a region having at least three-dimensional axes and which displays a value group related to a target tissue in such a manner that a value group related to a specific target serving as a reference is always displayed in a same position, and then causing a display to display the scatter diagram.
